(19) United States
(12) Patent Application Publication
(10) Pub. No.: US 2021/0393907 A1
(43) Pub. Date: Dec. 23, 2021

(54) SUCTIONING DEVICE FOR VENTILATED PATIENTS AND ASSOCIATED SYSTEM AND METHOD

(71) Applicant: HACKENSACK MERIDIAN HEALTH, INC., Edison, NJ (US)

(72) Inventors: Nasim Ahmed, Toms River, NJ (US); Abimbola Pratt, Asbury Park, NJ (US); Marco Corrales, Long Branch, NJ (US)

(73) Assignee: HACKENSACK MERIDIAN HEALTH, INC., Edison, NJ (US)

(21) Appl. No.: 17/349,622

(22) Filed: Jun. 16, 2021

Related U.S. Application Data
(60) Provisional application No. 63/041,534, filed on Jun. 19, 2020.

(51) Int. Cl. A61M 16/04; A61M 16/08; A61M 16/20; A61M 16/00

(52) U.S. Cl. CPC A61M 16/0463; A61M 16/0475; A61M 16/0833; A61M 2016/0027; A61M 16/0003; A61M 2205/3553; A61M 16/208

(57) ABSTRACT

An exemplary suction catheter is provided. The suction catheter includes a body defining a tubular structure, the body including a proximal end and a distal end on opposing sides of the body. The suction catheter includes a first suction hole formed in the body and spaced from the distal end by a first distance, a second suction hole formed in the body and spaced from the distal end by a second distance, a third suction hole formed in the body and spaced from the distal end by a third distance, and a fourth suction hole formed in the body and spaced from the distal end by a fourth distance. The fourth distance is greater than the first, second and third distances, the third distance is greater than the first and second distances, and the second distance is greater than the first distance.

### CROSS-REFERENCE TO RELATED APPLICATIONS

This application claims the benefit of priority to provisional application 63/041,534, filed on Jun. 19, 2020, the content of which is incorporated by reference in its entirety.

### BACKGROUND

Intubation of a patient in the medical industry is generally performed to assist the patient with breathing during anesthesia, sedation, and/or severe illness. During intubation, an endotracheal tube is inserted through the mouth of the patient and into the airway. The endotracheal tube is then connected to a ventilator that pushes air into the lungs of the patient through the endotracheal tube to assist in breathing. Secretions can pool in the airway tracts while the patient is intubated and, because the coughing reflex is blunted during intubation, the patient is unable to clear secretions on their own. Such secretions are periodically suctioned out by manually inserting a suction catheter down the endotracheal tube until the airway is cleared, and subsequently removing the suction catheter. If additional suctioning is needed at a later time, a new suction catheter is generally used. Traditional suctioning of the secretions therefore necessitates the physical presence of a medical professional at the bedside of the patient to perform the suctioning.

The necessity of being physically present to suction the secretions can be problematic when treating highly contagious patients. As an example, intubation has been used at an increasing frequency for COVID-19 patients having severe respiratory issues. Although healthcare providers generally wear personalized protective equipment and face masks (e.g., N95E masks) to protect themselves while working with patients, due to shortages in or limits of protection provided by personalized protective equipment, there is still a risk of exposure to an aerosolized virus.

### SUMMARY

In accordance with some embodiments of the present disclosure, an exemplary suctioning device is provided. The suctioning device provides an automated means for suctioning secretions pooling in the airway tracts of an intubated patient. The suctioning device is passed through an endotracheal tube and can be maintained within the endotracheal tube while the patient is intubated. Thus, rather than removing and replacing the suction catheter after each suctioning operation, a single suction catheter can remain in position for multiple uses. The distal end of the suction catheter can be maintained just beyond the distal end of the endotracheal tube to ensure proper suctioning of secretions. The suction catheter of the suctioning device can include additional openings along the length of the suction catheter to allow for suctioning of secretions when the patient is proned during intubation. The suctioning device therefore provides separation between the medical professional and the highly contagious patient during suctioning while the patient is intubated, reducing potential risks for medical professionals. The automated suctioning device can thereby prevent or reduce disease transfer and can improve efficiency of the suctioning process.

In accordance with embodiments of the present disclosure, an exemplary suction catheter is provided. The suction catheter includes a body defining a tubular structure, the body including a proximal end and a distal end on opposing sides of the body. The suction catheter includes a first suction hole formed in the body and spaced from the distal end by a first distance. The suction catheter includes a second suction hole formed in the body and spaced from the distal end by a second distance. The suction catheter includes a third suction hole formed in the body and spaced from the proximal end by a third distance. The suction catheter includes a fourth suction hole formed in the body and spaced from the proximal end by a fourth distance. The third distance is dimensioned greater than the fourth distance, and the second distance is dimensioned greater than the first distance.

The proximal end can be configured to be coupled to a suction machine for suctioning secretions in a trachea of a patient through a hollow interior of the body. The distal end can be configured to be passed into a trachea of a patient for suctioning secretions from the trachea through a hollow interior of the body. The first and fourth suction holes can be formed on one side of the body and the second and third suction holes can be formed on an opposing side of the body. The second and third suction holes can be radially oriented about 180° relative to the first and fourth suction holes. In some embodiments, the first distance can be about 5 mm or about 6 mm, the second distance can be about 1 cm or about 1.2 cm, the third distance can be about 10 mm, and the fourth distance can be about 4 mm.

In accordance with embodiments of the present disclosure, an exemplary suctioning device is provided. The suctioning device includes an endotracheal tube including a proximal end and a distal end. The suctioning device includes a suction catheter including a proximal end and a distal end. The suction catheter can be detachably secured relative to the endotracheal tube. The suctioning device includes a suction machine coupled to the suction catheter, a timer electrically connected to the suction machine, and a remote control actuator communicatively connected to the timer. The remote control actuator can be disposed remotely from the suction catheter. Actuation of the remote control actuator into an ON position actuates the timer into an ON position which, in turn, actuates the suction machine into an ON position to perform a suctioning procedure using the suction catheter.

The suction catheter can be detachably secured relative to the endotracheal tube such that the distal end of the suction catheter partially extends beyond the distal end of the endotracheal tube by a distance. In some embodiments, the distance can be about 5 mm. The proximal end of the endotracheal tube can be coupled to a T-connector, with one extension of the T-connector coupled to a ventilator to introduce air into the endotracheal tube through the T-connector. The suctioning device can include a connector coupled to the proximal end of the suction catheter. The connector can couple the suction catheter to tubing associating with the suction machine. In some embodiments, the connector can include a one-way valve to regulate a direction of secretion passage through the connector.

The suction catheter can include a first suction hole formed in the body and spaced from the distal end of the suction catheter by a first distance. The suction catheter can include a second suction hole formed in the body and spaced from the distal end of the suction catheter by a second distance. The suction catheter can include a third suction hole formed in the body and spaced from the proximal end of the suction catheter by a third distance. The suction catheter can include a fourth suction hole formed in the body and spaced from the proximal end of the suction catheter by a fourth distance. The third distance is dimensioned greater than the fourth distance, and the second distance is dimensioned greater than the first distance. In some embodiments, the first and fourth suction holes can be formed on one side of the body and the second and third suction holes can be formed on an opposing side of the body and can be radially oriented about 180° relative to the first and fourth suction holes.

In accordance with embodiments of the present disclosure, an exemplary suctioning device is provided. The suctioning device includes an endotracheal tube including a proximal end and a distal end. The suctioning device includes a suction catheter including a proximal end and a distal end. The suction catheter can be detachably secured relative to the endotracheal tube. As used herein, detachably secured refers to a semi-permanently positioned suction catheter within the endotracheal tube, with the position of the suction catheter maintained relative to the endotracheal tube without physical input from a medical professional. The detachably secured configuration allows for the suction catheter to remain in the selected position for multiple cycles of suctioning of the patient without physically moving the suction catheter relative to the endotracheal tube. However, the detachable nature of the coupling allows for the suction catheter to be removed from the endotracheal tube for the purpose of disassembling the suction catheter from the endotracheal tube. The suctioning device includes a suction machine coupled to the suction catheter. The suctioning device includes one or more sensors disposed at or near the distal end of the suction catheter. The one or more sensors can be configured to detect a change in a condition within or around the suction catheter. In some embodiments, the sensor(s) can be a balloon sensor configured to detect changes in pressure and/or flow from secretions within or around the suction catheter. The suctioning device includes a processing device in communication with the one or more sensors to define a feedback control loop. The processing device can be configured to receive data associated with the change in the condition within or around the suction catheter from the one or more sensors and automatically regulates operation of the suction machine based on the received data from the one or more sensors.

In some embodiments, the one or more sensors can be pressure sensors and the condition is pressure. In some embodiments, the one or more sensors can be fluid sensors and the condition is accumulation of secretions. In some embodiments, the suction catheter can be detachably secured relative to the endotracheal tube such that the distal end of the suction catheter partially extends beyond the distal end of the endotracheal tube by a distance of about 5 mm.

The suction catheter can include a first suction hole (e.g., a first suction hole from the distal end) formed in the body and spaced from the distal end of the suction catheter by a first distance. The suction catheter can include a second suction hole (e.g., a second suction hole from the distal end) formed in the body and spaced from the distal end of the suction catheter by a second distance. The suction catheter can include a third suction hole (e.g., a third suction hole formed from the distal end and a second suction hole formed from the proximal end) formed in the body and spaced from the proximal end of the suction catheter by a third distance. The suction catheter can include a fourth suction hole (e.g., a fourth suction hole formed from the distal end and a first suction hole formed from the proximal end) formed in the body and spaced from the proximal end of the suction catheter by a fourth distance. The third distance is dimensioned greater than the fourth distance, and the second distance is dimensioned greater than the first distance. The first and second suction holes assist in suctioning secretions at or near the distal end of the endotracheal tube. The third and fourth suction holes assist in suctioning secretions that move towards the proximal end of the endotracheal tube, e.g., when the patient is proned (e.g., flipping the patient over on their chest, resulting in secretions gravitating towards the proximal end of the endotracheal tube).

In accordance with embodiments of the present disclosure, an exemplary method of suctioning and reducing exposure of a healthcare practitioner to infectious aspirate is provided. The method includes detachably securing a suction catheter relative to an endotracheal tube. The endotracheal tube and the suction catheter each include proximal and distal ends, and the suction catheter is coupled to a suction machine. The method includes actuating a remote control actuator into an ON position to actuate a timer electrically connected to the suction machine into an ON position. The remote control actuator is communicatively connected to the timer and is disposed remotely from the suction catheter. Actuation of the timer into the ON position actuates the suction machine into an ON position to perform a suctioning procedure using the suction catheter.

In accordance with embodiments of the present disclosure, an exemplary method of suctioning and reducing exposure of a healthcare practitioner to infectious aspirate is provided. The method includes detachably securing a suction catheter relative to an endotracheal tube. The endotracheal tube and the suction catheter each include proximal and distal ends, and the suction catheter is coupled to a suction machine. The method includes detecting a change in a condition within or around the suction catheter with one or more sensors disposed at or near the distal end of the suction catheter. The method includes receiving data associated with the change in the condition within or around the suction catheter from the one or more sensors at a processing device. The method includes automatically regulating operation of the suction machine with the processing device based on the received data from the one or more sensors.

In accordance with embodiments of the present disclosure, an exemplary suctioning device is provided. The suctioning device includes an endotracheal tube including a proximal end and a distal end, and a suction catheter. The suction catheter includes a body defining a tubular structure with a proximal end and a distal end on opposing sides of the body. The suction catheter is detachably secured relative to the endotracheal tube such that the distal end of the suction catheter partially extends beyond the distal end of the endotracheal tube. The suction catheter includes a first suction hole formed in the body and spaced from the distal end by a first distance. The suction catheter includes a second suction hole formed in the body and spaced from the distal end by a second distance. The suction catheter includes a third suction hole formed in the body and spaced from the proximal end by a third distance. The suction catheter includes a fourth suction hole formed in the body and spaced from the proximal end by a fourth distance. In some embodiments, the third distance is dimensioned greater than the fourth distance, and the second distance is dimensioned greater than the first distance. The suctioning device includes a suction machine coupled to the suction catheter. One or more sensors are disposed at or near the distal end of the suction catheter, the one or more sensors configured to detect a change in a condition within or around the suction catheter. The suctioning device includes a processing device in communication with the one or more sensors to define a feedback control loop. The processing device is configured to receive data associated with the change in the condition within or around the suction catheter from the one or more sensors and automatically regulate operation of the suction machine based on the received data from the one or more sensors.

In accordance with embodiments of the present disclosure, an exemplary method of suctioning and reducing exposure of a healthcare practitioner to infectious aspirate is provided. The method includes detachably securing a suction catheter relative to an endotracheal tube. The endotracheal tube and the suction catheter each include proximal and distal ends. The suction catheter is detachable secured to the endotracheal tube such that the distal end of the suction catheter partially extends beyond the distal end of the endotracheal tube. The suction catheter is coupled to a suction machine, The suction catheter includes a first suction hole formed in the body and spaced from the distal end by a first distance. The suction catheter includes a second suction hole formed in the body and spaced from the distal end by a second distance. The suction catheter includes a third suction hole formed in the body and spaced from the proximal end by a third distance. The suction catheter includes a fourth suction hole formed in the body and spaced from the proximal end by a fourth distance. The third distance is dimensioned greater than the fourth distance, and the second distance is dimensioned greater than the first distance. The method includes detecting a change in a condition within or around the suction catheter with one or more sensors disposed at or near the distal end of the suction catheter. The method includes receiving data associated with the change in the condition within or around the suction catheter from the one or more sensors at a processing device to define a feedback control loop. The method includes automatically regulating operation of the suction machine with the processing device based on the received data from the one or more sensors.

Any combination and/or permutation of embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed suction catheter, reference is made to the accompanying figures, wherein:

FIG. 1 is a diagrammatic side view of an exemplary suctioning device of the present disclosure including a remote control actuator;

FIG. 2 is a diagrammatic side view of a suction catheter of an exemplary suctioning device of the present disclosure;

FIG. 3 is a diagrammatic side view of an exemplary suctioning device of the present disclosure including a feedback control system; and

FIG. 4 is a diagrammatic side view of a suctioning device of the present disclosure including a one-way valve.

### DETAILED DESCRIPTION

Various terms relating to the devices, methods and other aspects of the present disclosure are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “one or more” as used herein is defined as any whole integer greater than the number 1, e.g., 2, 3, 4, 5 or more. The term “plurality” as used herein is defined as any amount or number greater or more than 1. In some embodiments, the term “plurality” means 2, 3, 4, 5, 6 or more.

### Anatomical Terms

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

The term ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

The term “prone” denotes the position of the body when lying face downward. The term “supine” denotes the body when lying face upward.

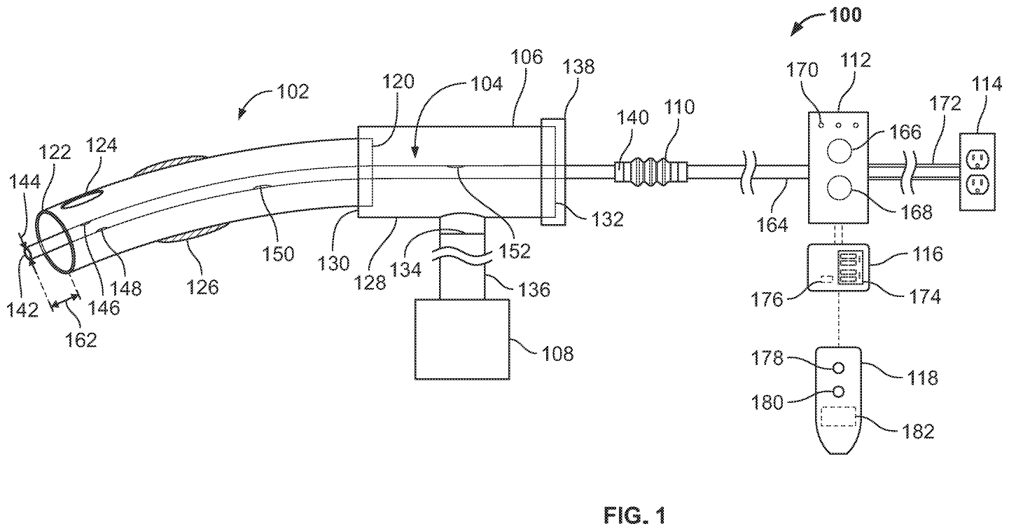

FIG. 1 is a diagrammatic side view of an exemplary suctioning device 100 (e.g., a suctioning system) of the present disclosure. The suctioning device 100 includes an endotracheal tube 102, a suction catheter 104 disposed at least partially within the endotracheal tube 102, and a T-connector 106. The suctioning device 100 includes a connector 110, and a suction machine 112 electrically connected to a power source 114. In the embodiment of FIG. 1, the suctioning device 100 can be used with a ventilator 108. In the embodiment of FIG. 1, the suctioning device 100 can include a timer 116 electrically connected in a wired and/or wireless manner to the suction device or machine 112, and a remote control actuator 118 electrically connected in a wireless manner to the timer 116.

The suctioning device 100 can incorporate endotracheal tubes 102 of various sizes, including but not limited to the endotracheal tubes listed in Table 1 below. Table 1 provides non-limiting examples of endotracheal tubes. The endotracheal tube 102 includes a proximal end 120 and an opposing distal end 122, and generally defines a tubular configuration with a hollow interior. A side opening 124 can be formed in the endotracheal tube 102 at or near the distal end 122, and a balloon cuff 126 (shown in cross-section for clarity of other components of the suctioning device 102) can be used on the exterior of the endotracheal tube 102 to provide a seal with the airway.

TABLE 1
Endotracheal Tube Sizes

| | | Inner | Outer |
|---|---|---|---|
| | | Diameter | Diameter |
| Patient | Length (cm) | (mm) | (mm) |
| Neonate | 7.5-10.5 (oral)/9.0-13.0 (nasal) | 2.5-3.0 | 3.4-4.2 |
| Infant | 11.0-12.5 (oral)/14.0-15.5 (nasal) | 3.5-4.5 | 4.8-6.2 |
| Child (2-10 yrs) | 12.5-16.0 (oral)/15.5-19.5 (nasal) | 4.5-6.5 | 6.2-8.8 |
| Teenager (11-18 yrs) | 16.0-22.0 (oral)/19.5-25.0 (nasal) | 6.5-8.5 | 8.8-11.6 |
| Adult Female | 17.0-21.0 (oral)/20.0-24.0 (nasal) | 7.0-8.0 | 9.6-11.0 |
| Adult Male | 21.0-23.0 (oral)/24.0-26.0 (nasal) | 8.0-9.5 | 11.0-13.0 |

The proximal end 120 of the endotracheal tube 102 can be coupled to the T-connector 106. The T-connector 106 includes a top extension 128 with opposing ends 130, 132. In some embodiments, the top extension 128 can have a length of about 2 inches between the ends 130, 132. A bottom extension 134 extends perpendicularly relative to the top extension 128, with the interior passages of the top extension 128 and the bottom extension 134 fluidly connected. The ventilator 108 is coupled to the bottom extension 134 of the T-connector 106 by tubing 136 such that operation of the ventilator 108 allows for passage of air through the T-connector 106 and the endotracheal tube 102 to assist the patient with breathing. The end 132 of the T-connector 106 can include an adapter fitting 138 to accommodate the difference in diameter between the opening at the end 132 and the outer diameter of the suction catheter 104. In particular, the adapter fitting 138 creates a seal around the end 132 and the suction catheter 104 to prevent leakage of airflow during operation of the ventilator 108.

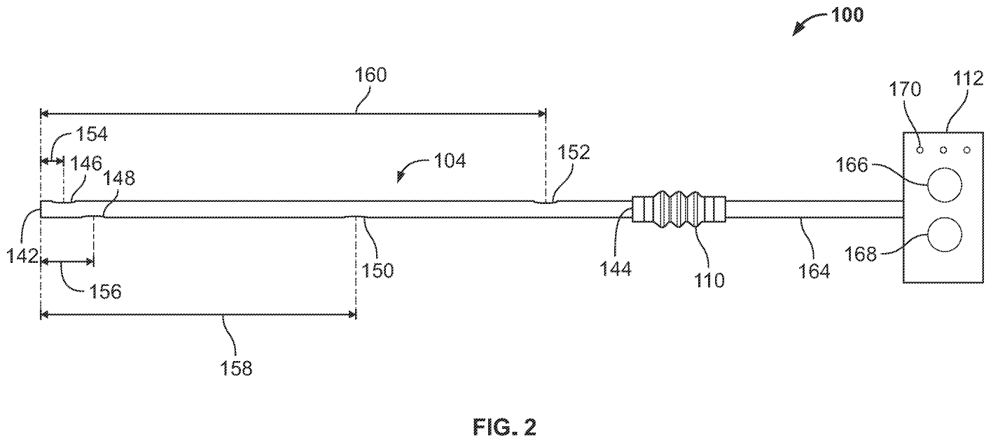

The suction catheter 104 defines a cylindrical, tubular body including a proximal end 140 and a distal end 142. In some embodiments, the suction catheter 104 can incorporate a three-way adapter at the proximal end 140, the three-way adapter accommodating air, suction and flushing. In some embodiments, a radiopaque marker can be included along the length of the suction catheter 104. The suction catheter 104 shown in FIG. 2 is a size 10 Fr catheter having an outer diameter 144 of about 0.131 inches and configured to function with a size 8 endotracheal tube 102. However, it should be understood that suction catheters 104 of different sizes can be incorporated into and used with differently sized endotracheal tubes 102 of the suctioning device 100. As discussed herein, the unit “Fr” refers to the French size scale which is a measure of the outer diameter of a catheter tube. 1 Fr is defined as ⅓ of a mm (3.333×10⁻⁴ meters). Therefore a 10 Fr catheter has a 3.33 mm diameter (i.e., a diameter in mm multiplied by 3). The suction catheters 104 discussed herein can come in different diameters and/or different lengths.

In some embodiments, Equation 1 below can be used to determine the proper Fr size of the suction catheter 104 to be used based on the endotracheal tube 102 size.

Fr=(ETT Size−1)×2 (1)

In Equation 1, ETT Size is the endotracheal tube 102 size in mm. Equation 1 can therefore be used to approximate the Fr size of the suction catheter 104 to be used. With reference to FIGS. 1 and 2, the suction catheter 104 includes four suction holes 146-152 formed in the body and defining passages extending from the hollow interior of the body to the exterior of the suction catheter 104. As shown in FIG. 2, each of the suction holes 146-152 can be spaced relative to each other and from the distal end 142.

For example, the first suction hole 146 can be spaced from the distal end 142 by a distance 154 (e.g., about 4 mm, about 5 mm, about 6 mm, about 7 mm, about 4-7 mm, about 5-6 mm, or the like), the second suction hole 148 can be spaced from the distal end 142 by a distance 156 (e.g., about 1 cm, about 1.1 cm, about 1.2 cm, about 1.3 cm, about 1.4 cm, about 1-1.4 cm, about 1-1.3 cm, about 1-1.2 cm, or the like), the third suction hole 150 can be spaced from the distal end 142 by a distance 158 (e.g., about 23 cm, about 24 cm, about 25 cm, about 26 cm, about 27 cm, about 28 cm, about 23-28 cm, about 24-27 cm, about 25-26 cm, or the like), and the fourth suction hole 152 can be spaced from the distal end 142 by a distance 160 (e.g., about 35 cm, about 36 cm, about 37 cm, about 38 cm, about 39 cm, about 40 cm, about 35-40 cm, about 36-39 cm, about 37-38 cm, or the like).

The third suction hole 150 (e.g., the second suction hole from the proximal end 144) can be spaced, e.g., about 8 mm, about 9 mm, about 10 mm, about 11 mm, about 12 mm, about 8-12 mm, about 9-11 mm, about 8-11 mm, about 8-10 mm, about 9-12 mm, about 9-10 mm, or the like, from the proximal end 144 of the suction catheter. The fourth suction hole 152 (e.g., the first suction hole from the proximal end 144) can be spaced, e.g., about 2 mm, about 3 mm, about 4 mm, about 5 mm, about 6 mm, about 7 mm, about 8 mm, about 2-8 mm, about 3-7 mm, about 4-6 mm, about 2-7 mm, about 2-6 mm, about 2-5 mm, about 2-4 mm, about 2-3 mm, about 3-8 mm, about 4-8 mm, about 5-8 mm, about 6-8 mm, about 7-8 mm, or the like, from the proximal end 144 of the suction catheter 104. In some embodiments, the fourth suction hole 152 can be spaced about 2 cm from the end 130 of the T-connector 106 after assembly of the suctioning device 100. However, it should be understood that the distances of the suction holes 146-152 relative to the proximal and distal ends 142, 144 (or the T-connector 106) can be varied based on the overall length of the suction catheter 104. In some embodiments, the suction catheter 104 can include three suction holes 146, 148, 150, with only one suction hole 150 at or near the proximal end 144. In some embodiments, the length of the suction catheter 104 can be selected such that after assembly with the T-connector 106, the distal end 142 of the suction catheter 104 is about 12 inches or about 14 inches away from the end 130 of the T-connector 106. The spacing of the suction holes 146-152 is provided as an example for a size 10 Fr suction catheter 104 and a size 8 endotracheal tube 102. As a further example, for a size 7 endotracheal tube 102, the distal suction holes (e.g., suction holes 146, 148) can be spaced at the substantially same distance from the distal end 142, while the proximal suction holes (e.g., suction holes 150, 152) can each be spaced 2 cm closer to the distal end 142 (e.g., at 24 cm and 36 cm, respectively). Each of the suction holes 146-152 can define a substantially oblong or oval configuration. In some embodiments, each of the suction holes 146-152 can define substantially equal dimensions. In some embodiments, the suction hole 146 can define a length of 2.5 mm and a width of 2 mm, the suction hole 148 can define a length of 2.0 mm and a width of 1.8 mm, the suction hole 150 can define a length of 25 mm and a width of 2.0 mm, and the suction hole 152 can define a length of 2.5 mm and a width of 2.0 mm. The different dimensions of the suction holes 146-152 allow for suctioning of secretions or fluids having different viscosities. For example, the smaller suction holes can be used to collect thin secretions, while the slightly larger suction holes can be used for thicker mucous secretions. The suction catheter 104 can thereby function to collection secretions having different consistencies. In some embodiments, the radial orientation of the suction holes 146-152 can be different relative to each other. For example, the suction holes 146, 152 can be disposed at one side of the suction catheter 104, and the suction holes 148, 150 can be radially offset by about 180° from the suction holes 146, 152 (e.g., the opposing side of the suction catheter 104). The radially offset orientation of the suction holes 146-152 ensures that secretions on opposing sides of the suction catheter 104 can be collected, resulting in an efficient and effective operation.

In some embodiments, the radial offset of each of the suction holes 146-152 can be different. In some embodiments, additional suction holes can be added along the length of the suction catheter 104. In some embodiments, additional suction holes can be added at the proximal end 144 of the suction catheter 104. In some embodiments, the additional suction holes can be radially offset by 90° from the suction holes 146-152 to provide additional suction on the sides of the suction catheter 104. Although shown with two suction holes 150, 152 at or near the proximal end 144, in some embodiments, the suction catheter 104 can include one or more suction holes at the proximal end 144. In some embodiments, the suction catheter 104 can be without suction holes 150, 152 at the proximal end 144.

As shown in FIG. 1, the suction catheter 104 is passed through the T-connector 106 and the endotracheal tube 102 such that the distal end 142 of the suction catheter 104 extends just beyond the distal end 122 of the endotracheal tube 102 by a distance 162. In some embodiments, an automatic spring mechanism can be used to place and retrieve the suction catheter 102 in the endotracheal tube 102. In some embodiments, placement and retrieval of the suction catheter 102 from the endotracheal tube 102 can be performed periodically. In some embodiments, the distance 162 can be about, e.g., 2-10 mm, 3-9 mm, 4-8 mm, 5-7 mm, 2-8 mm, 2-6 mm, 3-5 mm, 2 mm, 3 mm, 4 mm, 5 mm, 6 mm, 7 mm, 8 mm, 9 mm, 10 mm, or the like.

The protrusion of the distal end 142 of the suction catheter 104 relative to the distal end 122 of the endotracheal tube 102 ensures that the suction catheter 104 is disposed in the trachea where secretions can collect, providing an effective position for suctioning. The protruding distal end 142 also ensures that secretions can be removed from the trachea before traveling into the endotracheal tube 102 and upwards along the endotracheal tube 102, resulting in a more efficient suctioning process and improved comfort for the patient. The overall length of the suction catheter 104 can therefore be selected based on the length of the endotracheal tube 102 used to achieve the minimal protrusion of the distal end 142 from the distal end 122 of the endotracheal tube 102. In comparison, traditional suction catheters are generally significantly longer in length than the endotracheal tube 102 since the suction catheter is manually fed by the medical professional to the trachea for suctioning. In some embodiments, a sterile transparent plastic sheath (not shown for clarity) can cover the entire length of the suction catheter 104.

The suction catheter 104 can be coupled to the T-connector 106 and/or the endotracheal tube 102 to remain in the position shown in FIG. 1 for automated suctioning of the patient. The different positions and/or radial orientations of the suction holes 146-152 ensures that secretions are suctioned along the length of the suction catheter 104. In addition, the proximal suction holes (e.g., suction holes 150, 152) provide for suctioning of secretions that may be redistributed or moved within the trachea of the patient during proning of the patient. For example, traditional suction catheters generally only include two holes at the distal end for manual feeding of the suction catheter through the endotracheal tube 102 and suctioning along the path as the suction catheter is inserted into the trachea or removed from the trachea. Due to the manual introduction, removal and reintroduction of the traditional suction catheter, such suction catheters do not include additional suction holes along the length of the suction catheter.

In contrast, the suction catheter 104 discussed herein remains in the endotracheal tube 102 for multiple cycles of suctioning and until removal of the suction catheter 104 is desired. The suction catheter 104 can thereby remain in place and use of the suction catheter 104 can be prolonged as compared to traditional suction catheters, reducing costs associated with the procedure or care of the patient. As such, the suction holes 146, 148 at or near the distal end 142 provide for suctioning secretions built up at the trachea of the patient. If the patient is proned while the suction catheter 104 remains in place, the position of the secretions may shift, e.g., rise higher along the endotracheal tube 102. For example, if a patient is flipped, the head/chest of the patient are positioned downward, gravitating the secretions towards the chest of the patient and the proximal end 140 of the endotracheal tube 102. The suction holes 150, 152 therefore provide additional openings through which secretions can be suctioned or captured into the suction catheter 104.

The proximal end 140 of the suction catheter 104 can extend from the end 132 of the T-connector 106 and can be coupled to a connector 110. In some embodiments, the connector 110 can act as a sealed adapter for connecting the suction catheter 104 to the suction machine 112 via tubing 164. The suction machine 112 provides the suction force through the suction catheter 104 for capturing the secretions. The suction machine 112 can include visual indicators 166, 168 that provide the mode of operation of the suction machine 112. For example, the visual indicators 166, 168 can be green and red light-emitting diodes (LEDs) for indicating the ON and OFF operation status of the suction machine 112. The suction machine 112 can include one or more additional visual indicators 170 (e.g., LEDs) that indicate a specific status of the suction machine 112, such as the speed/force of suction, whether the secretion collection canister is full, combinations thereof, or the like. The suction machine 112 is electrically connected via cable 172 to the power source 114 (e.g., an electrical outlet).

In the embodiment of FIG. 1, the timer 116 can be electrically connected to the suction machine 112 in a wired and/or wireless manner. In some embodiments, the timer 116 can be electrically connected to the power source 114. The timer 116 includes an integrated circuit or chip 176 capable of receiving and transmitting signals, and capable of timed operation. The timer 116 can include a user interface 174 (e.g., a graphical user interface) that can visually indicate the time left in an operation cycle, the time until initiation of the next cycle, information on previous cycles, or the like. The user interface 174 can also provide means for programming the timer 116 for the desired operation, such as the length of an ON operation cycle and the frequency of such ON operation cycles.

The remote control actuator 118 is electrically connected in a wireless manner to the timer 116, and is in electrical communication with the timer 116. Specifically, the actuator 118 is capable of transmitting control signals to the timer 116. The actuator 118 includes an ON switch or button 178 and an OFF button 180 for actuating the timer 116 into an ON or OFF position, respectively. The actuator 118 can include a user interface 182 for visually displaying information associated with operation of the timer 116. In operation, the timer 116 can be electrically connected to and disposed near the suction machine 112, which is positioned in the room of the patient. The actuator 118 can be remotely held by a medical professional (e.g., in a different room than the room of the patient).

The actuator 118 can be actuated into the ON position to actuate the timer 116 into the ON position. The timer 116 can, in turn, initiate operation of the suction machine 112 to suction secretions. In some embodiments, the timer 116 can operate in the ON position for a predetermined period of time, e.g., 5 seconds, 6 seconds, 7 seconds, 8 seconds, 9 seconds, 10 seconds, 11 seconds, 12 seconds, 13 seconds, 14 seconds, 15 seconds, 5-15 seconds, 6-14 seconds, 7-13 seconds, 8-12 seconds, 9-11 seconds, 5-13 seconds, 5-10 seconds, 10-15 seconds, 15 seconds or less, or the like, and automatically actuate into the OFF position to turn off the suction machine 112 without input from the medical professional. Generally, it is preferable for the suctioning procedure to be performed only up to 15 seconds prior to actuating the suction machine 112 into the OFF position to prevent continuous suctioning. If additional suctioning is desired, the medial professional can again actuate the actuator 118 into the ON position remotely to reset automatic suctioning for another 15 seconds (or another predetermined period of time below 15 seconds). In some embodiments, the medical professional can override the ON operation of the timer 116 by actuating the OFF button 180. The medical professional can thereby operate the suctioning procedure without exposure to a potentially contagious patient. The suction catheter 102 also remains sterile and capable of being used for multiple occasions because the suction catheter 102 is not removed from the patient after each use.

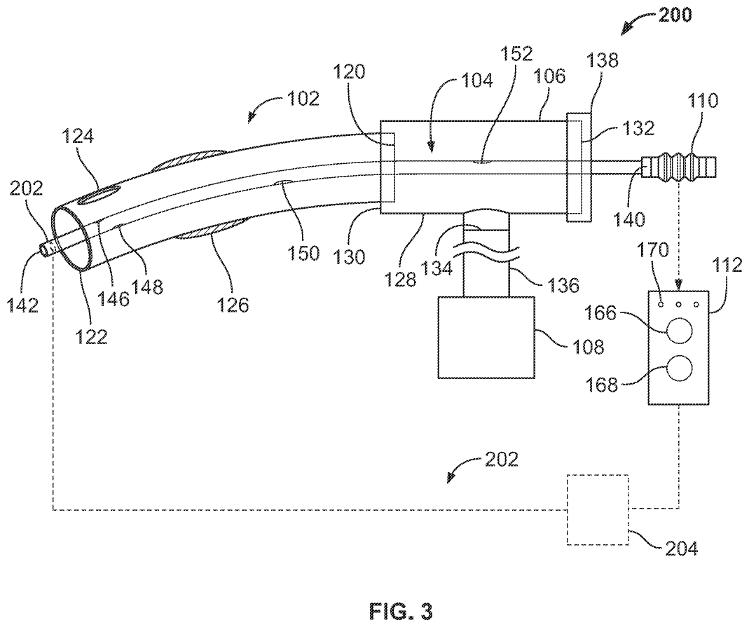

FIG. 3 is a diagrammatic side view of an exemplary suctioning device 200 of the present disclosure. The suctioning device 200 can be substantially similar in structure and/or function except for the distinctions noted herein. Therefore, like reference numbers refer to like structures. Rather than including a timer 116 and remote control actuator 118, the suctioning device 200 can include a feedback control system 202 for continuously monitoring for signs of secretion build-up at or around the suction catheter 104 and automatically actuating the suction machine 112 into the ON position upon detection of secretion build-up.

The suctioning device 200 can include one or more sensors 202 disposed along the length of the suction catheter 104. Although illustrated as including a sensor 202 at or near the distal end 142 of the suction catheter 104, it should be understood that the suctioning device 200 can include multiple sensors 202 disposed in a spaced relationship along the length of the suction catheter 104. For example, the suction catheter 104 can include a sensor 202 at the distal end 142, and also sensors 202 at or near each of the suction holes 146-152. In some embodiments, the sensor 202 can be, e.g., a pressure sensor, a fluid sensor, a displacement sensor, a light blockage sensor, or the like, configured to detect changes or fluctuations of pressure, for example, to determine the existence of secretion build-up around the suction catheter 104.

In some embodiments, the sensor 202 can be a balloon-type sensor configured to detect pressure applied to the sensor 202 by the secretions. For example, the inflated balloon can detect pressure from the secretions and can exert a force against the secretions to clear the path in the tubing, with detection of the secretions automatically initiating the suction machine 112 into the ON position. In some embodiments, the sensor 202 can detect a blockage of the suction catheter 102 and can transmit a signal to generate an alert to the medical professional. In some embodiments, the sensor 202 can detect if the suction pressure reaches or exceeds a predetermined maximum pressure considered safe for the patient, and actuates the suction machine 112 into the OFF position upon reaching the predetermined maximum pressure. In some embodiments, the sensor 202 can detect if the suction pressure reaches or exceeds a predetermined maximum pressure, identifies the elevated pressure as indicative of a blockage in the suction catheter 104, and actuates the suction machine 112 into the OFF position to prevent damage.

In some embodiments, the sensor 202 can detect if the motor for the suction machine 112 is operating at higher levels than is considered normal operation, identifies the elevated levels as indicative of a blockage in the suction catheter 104, and actuates the suction machine 112 into the OFF position to prevent damage. In such an embodiment, a visual and/or audio alert can be generated to the operator to indicate a potential blockage in the suction catheter 104. In some embodiments, the sensor 202 can be a fiber optic sensor capable of detecting and displaying secretion build-up in real-time (e.g., the thickness of the secretions, how persistent the secretions are, combinations thereof, or the like).

The sensor 202 is electrically connected in a wired and/or wireless manner to a processing device 204 which, in turn, is electrically connected to the suction machine 112. Upon detection of secretion build-up around the suction catheter 104 (in the form of a change in pressure, for example), the sensor 202 can transmit a signal to the processing device 204 regarding the detected change. The processing device 204 can determine whether the change in the detected characteristic or condition is equal to or above a predetermined value. If the change in the detected characteristic or condition is determined to be equal to or above a predetermined value, the processing device 204 can transmit a control signal to the suction machine 112 to actuate the suction machine 112 into the ON position to initiate the suctioning procedure.

In some embodiments, the suctioning procedure can be performed for a predetermined period of time, e.g., 5 seconds, 6 seconds, 7 seconds, 8 seconds, 9 seconds, 10 seconds, 11 seconds, 12 seconds, 13 seconds, 14 seconds, 15 seconds, 5-15 seconds, 6-14 seconds, 7-13 seconds, 8-12 seconds, 9-11 seconds, 5-13 seconds, 5-10 seconds, 10-15 seconds, 15 seconds or less, or the like, and subsequently the suction machine 112 can be automatically placed in the OFF position. In some embodiments, the suctioning procedure can be performed until the detected characteristic by the sensor 202 is reduced below the predetermined value. In some embodiments, one or more of the indicators 170 of the suction machine 112 can provide periodic alerts to the medical professional regarding the operational status of the suction machine 112 and/or the detected secretion levels in the patient.

The suctioning procedure can thereby be performed automatically upon detection of secretions by the sensor 202, and does not necessitate direct involvement of a medical professional. It should be understood that a manual override of the suctioning procedure would be available to the medical professional by actuation either via a remote control actuator 118 and/or via manual actuation of the buttons 166, 168 of the suction machine 112. In some embodiments, the sensor 202 can detect the need for suctioning and can generate an alert to the medical professional to use the remote control actuator 118 to remotely initiate the suctioning procedure (based on the embodiment of FIG. 1 incorporating the sensor 202 and processing device 204).

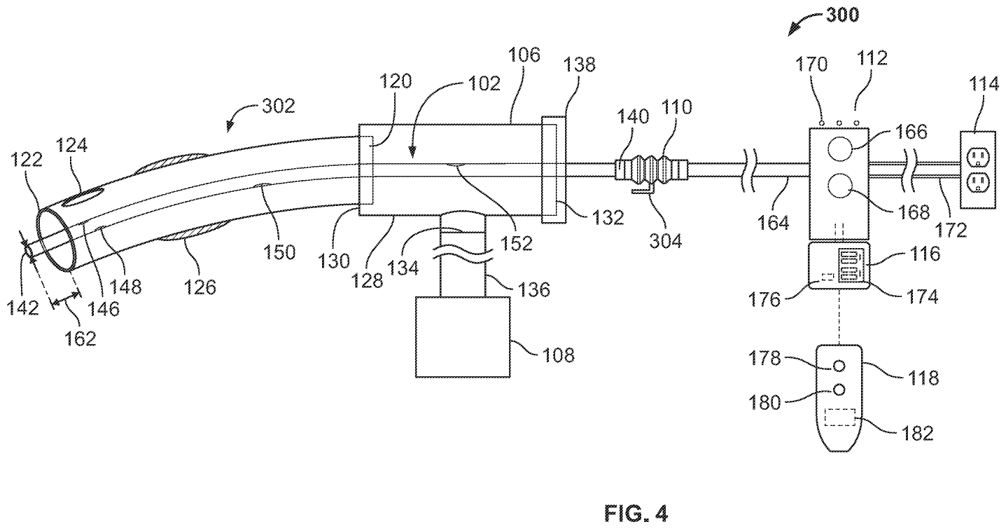

FIG. 4 is a diagrammatic side view of an exemplary suctioning device 300 of the present disclosure. The suctioning device 300 can be substantially similar in structure and/or function except for the distinctions noted herein. Therefore, like reference numbers refer to like structures. Rather than including an endotracheal tube 102, the suctioning device 300 can incorporate a tracheostomy tube 302. The tracheostomy tube 302 can include similar structures to the endotracheal tube 102 and, therefore, the reference numbers from FIG. 1 are shown in FIG. 4. The length of the suction catheter 104 and/or the position of the suction holes 146-152 can be adjusted to accommodate the length of the tracheostomy tube 302.

For example, the first suction hole 146 can be spaced from the distal end 142 by a distance 154 (e.g., about 4 mm, about 5 mm, about 6 mm, about 7 mm, about 8 mm, about 4-8 mm, about 5-7 mm, about 4-7 mm, about 4-6 mm, about 4-5 mm, about 5-8 mm, about 6-8 mm, about 7-8 mm, or the like), and the second suction hole 148 can be spaced from the distal end 142 by a distance 156 (e.g., about 1 cm, about 1.1 cm, about 1.2 cm, about 1.3 cm, about 1.4 cm, about 1.5 cm, about 1-1.5 cm, about 1-1.4 cm, about 1-1.3 cm, about 1-1.2 cm, about 1.1-1.5 cm, about 1.2-1.5 cm, about 1.3-1.5 cm, about 1.4-1.5 cm, or the like). Although FIG. 4 shows the suction catheter 104 with the proximal suction holes 150, 152, in some embodiments, the suction catheter 104 for the tracheostomy tube 302 can include only the distal suction holes 146, 148. Tracheostomy patients generally are not placed in a prone position and may not necessitate the suctioning at the proximal end 144. Therefore, the suction catheter 104 can either include each of suction holes 146-152, or can be effectively used with only suction holes 146, 148. The size of the suction catheter 104 can be, e.g., 8 Fr, 10 Fr, or the like. In some embodiments, the length of the suction catheter 104 (as measured from end 130 of the T-connector 106 after assembly with the T-connector 106 to the distal end 142 of the suction catheter 104) can be about 16 cm for a size 10 tracheostomy tube and about 15 cm for a size 6 tracheostomy tube.

The connector 110 can include a one-way valve 304 for controlling the direction of secretion passage through the connector 110. The connector 110 can connect the suction catheter 102 and the tube 164 leading to the suction machine 112. During suction, the valve 304 can be opened with the suction force from the suction machine 112. During the OFF operation of the suctioning machine 112, the valve 304 can automatically close during a lack of suctioning. The valve 304 can thereby reduce or prevent airflow through the suction catheter 102 in the direction of the distal end 142.

While exemplary embodiments have been described herein, it is expressly noted that these embodiments should not be construed as limiting, but rather that additions and modifications to what is expressly described herein also are included within the scope of the present disclosure. Moreover, it is to be understood that the features of the various embodiments described herein are not mutually exclusive and can exist in various combinations and permutations, even if such combinations or permutations are not made express herein, without departing from the spirit and scope of the present disclosure.

1. A suction catheter, comprising:
a body defining a tubular structure, the body including a proximal end and a distal end on opposing sides of the body;
a first suction hole formed in the body and spaced from the distal end by a first distance;
a second suction hole formed in the body and spaced from the distal end by a second distance;
a third suction hole formed in the body and spaced from the proximal end by a third distance; and
a fourth suction hole formed in the body and spaced from the proximal end by a fourth distance;
wherein the third distance is dimensioned greater than the fourth distance, and the second distance is dimensioned greater than the first distance.

2. The suction catheter of claim 1, wherein the proximal end is configured to be coupled to a suction machine for suctioning secretions in a trachea of a patient through a hollow interior of the body.

3. The suction catheter of claim 1, wherein the distal end is configured to be passed into a trachea of a patient for suctioning secretions from the trachea through a hollow interior of the body.

4. The suction catheter of claim 1, wherein the first and fourth suction holes are formed on one side of the body and the second and third suction holes are formed on an opposing side of the body.

5. The suction catheter of claim 4, wherein the second and third suction holes are radially oriented about 180° relative to the first and fourth suction holes.

6. The suction catheter of claim 1, wherein the first distance is about 5 mm or about 6 mm, the second distance is about 1 cm or about 1.2 cm, the third distance is about 10 mm, and the fourth distance is about 4 mm.

7. A suctioning device, comprising:
an endotracheal tube including a proximal end and a distal end;
a suction catheter including a proximal end and a distal end, the suction catheter detachably secured relative to the endotracheal tube;
a suction machine coupled to the suction catheter;
a timer electrically connected to the suction machine; and
a remote control actuator communicatively connected to the timer;
wherein the remote control actuator is disposed remotely from the suction catheter; and
wherein actuation of the remote control actuator into an ON position actuates the timer into an ON position which, in turn, actuates the suction machine into an ON position to perform a suctioning procedure using the suction catheter.

8. The suctioning device of claim 7, wherein the suction catheter is detachably secured relative to the endotracheal tube such that the distal end of the suction catheter partially extends beyond the distal end of the endotracheal tube by a distance.

9. The suctioning device of claim 8, wherein the distance is about 5 mm.

10. The suctioning device of claim 7, wherein the proximal end of the endotracheal tube is coupled to a T-connector, one extension of the T-connector coupled to a ventilator to introduce air into the endotracheal tube through the T-connector.

11. The suctioning device of claim 7, comprising a connector coupled to the proximal end of the suction catheter, the connector coupling the suction catheter to tubing associating with the suction machine.

12. The suctioning device of claim 11, wherein the connector includes a one-way valve to regulate a direction of secretion passage through the connector.

13. The suctioning device of claim 7, wherein the suction catheter includes:
a first suction hole formed in the body and spaced from the distal end of the suction catheter by a first distance;
a second suction hole formed in the body and spaced from the distal end of the suction catheter by a second distance;
a third suction hole formed in the body and spaced from the proximal end of the suction catheter by a third distance; and
a fourth suction hole formed in the body and spaced from the proximal end of the suction catheter by a fourth distance.

14. The suctioning device of claim 13, wherein the third distance is dimensioned greater than the fourth distance, and the second distance is dimensioned greater than the first distance.

15. The suction catheter of claim 13, wherein the first and fourth suction holes are formed on one side of the body and the second and third suction holes are formed on an opposing side of the body and are radially oriented about 180° relative to the first and fourth suction holes.

16. A suctioning device, comprising:
an endotracheal tube including a proximal end and a distal end;
a suction catheter including a proximal end and a distal end, the suction catheter detachably secured relative to the endotracheal tube;
a suction machine coupled to the suction catheter;
one or more sensors disposed at or near the distal end of the suction catheter, the one or more sensors configured to detect a change in a condition within or around the suction catheter; and
a processing device in communication with the one or more sensors to define a feedback control loop, the processing device configured to receive data associated with the change in the condition within or around the suction catheter from the one or more sensors and automatically regulate operation of the suction machine based on the received data from the one or more sensors.

17. The suctioning device of claim 16, wherein one or more sensors are pressure sensors and the condition is pressure, or wherein the one or more sensors are fluid sensors and the condition is accumulation of secretions.

18. The suctioning device of claim 16, wherein the suction catheter is detachably secured relative to the endotracheal tube such that the distal end of the suction catheter partially extends beyond the distal end of the endotracheal tube by a distance of about 5 mm.

19. The suctioning device of claim 16, wherein the suction catheter includes:
a first suction hole formed in the body and spaced from the distal end of the suction catheter by a first distance;
a second suction hole formed in the body and spaced from the distal end of the suction catheter by a second distance;
a third suction hole formed in the body and spaced from the proximal end of the suction catheter by a third distance; and
a fourth suction hole formed in the body and spaced from the proximal end of the suction catheter by a fourth distance.

20. The suctioning device of claim 19, wherein the third distance is dimensioned greater than the fourth distance, and the second distance is dimensioned greater than the first distance.

21. A method of suctioning, comprising:
detachably securing a suction catheter relative to an endotracheal tube, the endotracheal tube and the suction catheter each including proximal and distal ends, and the suction catheter coupled to a suction machine;
actuating a remote control actuator into an ON position to actuate a timer electrically connected to the suction machine into an ON position, wherein the remote control actuator is communicatively connected to the timer and is disposed remotely from the suction catheter;
wherein actuation of the timer into the ON position actuates the suction machine into an ON position to perform a suctioning procedure using the suction catheter.

22. A method of suctioning, comprising:
detachably securing a suction catheter relative to an endotracheal tube, the endotracheal tube and the suction catheter each including proximal and distal ends, and the suction catheter coupled to a suction machine;
detecting a change in a condition within or around the suction catheter with one or more sensors disposed at or near the distal end of the suction catheter;
receiving data associated with the change in the condition within or around the suction catheter from the one or more sensors at a processing device; and
automatically regulating operation of the suction machine with the processing device based on the received data from the one or more sensors.

23. A suctioning device, comprising:
an endotracheal tube including a proximal end and a distal end;
a suction catheter including:
a body defining a tubular structure with a proximal end and a distal end on opposing sides of the body, the suction catheter detachably secured relative to the endotracheal tube such that the distal end of the suction catheter partially extends beyond the distal end of the endotracheal tube,
a first suction hole formed in the body and spaced from the distal end by a first distance,
a second suction hole formed in the body and spaced from the distal end by a second distance,
a third suction hole formed in the body and spaced from the proximal end by a third distance, and
a fourth suction hole formed in the body and spaced from the proximal end by a fourth distance, wherein the third distance is dimensioned greater than the fourth distance, and the second distance is dimensioned greater than the first distance;
a suction machine coupled to the suction catheter;
one or more sensors disposed at or near the distal end of the suction catheter, the one or more sensors configured to detect a change in a condition within or around the suction catheter; and
a processing device in communication with the one or more sensors to define a feedback control loop, the processing device configured to receive data associated with the change in the condition within or around the suction catheter from the one or more sensors and automatically regulate operation of the suction machine based on the received data from the one or more sensors.

24. A method of suctioning, comprising:
detachably securing a suction catheter relative to an endotracheal tube, the endotracheal tube and the suction catheter each including proximal and distal ends, the suction catheter detachable secured to the endotracheal tube such that the distal end of the suction catheter partially extends beyond the distal end of the endotracheal tube, the suction catheter coupled to a suction machine, and the suction catheter including (i) a first suction hole formed in the body and spaced from the distal end by a first distance, (ii) a second suction hole formed in the body and spaced from the distal end by a second distance, (iii) a third suction hole formed in the body and spaced from the proximal end by a third distance, and (iv) a fourth suction hole formed in the body and spaced from the proximal end by a fourth distance, wherein the third distance is dimensioned greater than the fourth distance, and the second distance is dimensioned greater than the first distance;
detecting a change in a condition within or around the suction catheter with one or more sensors disposed at or near the distal end of the suction catheter;
receiving data associated with the change in the condition within or around the suction catheter from the one or more sensors at a processing device to define a feedback control loop; and
automatically regulating operation of the suction machine with the processing device based on the received data from the one or more sensors.
